(19) United States
(12) Patent Application Publication
(10) Pub. No.: US 2022/0389112 A1
(43) Pub. Date: Dec. 8, 2022

(54) TREATMENT OF MULTIPLE MYELOMA

(71) Applicant: NATIONAL UNIVERSITY OF IRELAND, GALWAY, Co. Galway (IE)

(72) Inventors: Michael O'DWYER, Galway (IE); John DALY, Co. Clare (IE); Sarkar SUBHASHIS, Galway (IE)

(73) Assignee: NATIONAL UNIVERSITY OF IRELAND, GALWAY, Co. Galway (IE)

(21) Appl. No.: 17/774,276

(22) PCT Filed: Nov. 6, 2020
(86) PCT No.: PCT/EP2020/081394

§ 371 Date: May 4, 2022

(30) Foreign Application Priority Data
Nov. 6, 2019 (GB) 1916150.4

(51) Int. Cl. C07K 16/28; A61K 31/203; C12N 9/24; A61K 35/17; A61P 35/00

(52) U.S. Cl. CPC C07K 16/2896; A61K 31/203; C12N 9/24; A61K 35/17; A61P 35/00

(57) ABSTRACT

Provided is an effective amount of a desialylation agent and an effective amount of an anti CD38 antibody for use in the treatment of multiple myeloma. This results in potentiated primary NK cell activity against the multiple myeloma cell. Also envisaged is a method of treating multiple myeloma in a subject in need thereof.

### BACKGROUND OF THE INVENTION

Multiple Myeloma (MM) is a cancer that forms in plasma cells. Normal plasma cells are a type of white blood cell that make antibodies. MM causes cancer cells to accumulate in the subject's bone marrow.

Evading Natural Killer (NK) cell-mediated immunosurveillance is key to the development of MM in a subject. Abnormal cell surface sialylation is considered a hallmark of cancer, including MM, and the current inventors have implicated hypersialylation in disease progression of MM. EP2906952 discloses that ST3GAL6, sialyltransferase which catalyses the transfer of sialic acid from cytidine 5-prime monophospho-N-acetylneuraminic acid (CMP-NeuAc) to terminal positions of glycoprotein and glycolipid carbohydrate groups, has an important role in MM disease biology. It has been suggested that sialyation plays a role in MM cell adhesion and migration. Hypersialylation of cells can lead to increased immune evasion and tumour invasiveness.

CD38 is a well validated target in the treatment of multiple myeloma. CD38 is expressed at high levels on myeloma cells and, to a lesser extent, on immune effector cells, including natural killer (NK) cells. Anti-CD38 monoclonal antibodies (for example, Daratumumab; Plesner et al., Front. Immuno., 2018; 9; 1228) are one type of therapeutic treatment available for MM patients. This antibody is well tolerated in patients and has shown to be effective in pre-treated relapsed and refractory MM. However, not all patients respond well, and some patients develop resistance. Mechanisms of Daratumumab resistance include upregulation of the complement inhibitory proteins, CD55/CD59, loss or dysfunction of innate effector cells (NK depletion (fratricide) and/or M2 polarization of macrophages) and an impaired adaptive T cell response, including major histocompatibility complex (MHC) class I down regulation, increase in programmed cell death protein 1 (PD-1), lymphocyte-activation gene 3 (LAG3) and TIGIT (T cell immunoreceptor with Ig and ITIM domains) immune checkpoints on T cells. Treatment with Daratumumab can induce a rapid reduction of CD38 expression on MM cells and a certain level of CD38 expression is required for Daratumumab induced antibody-dependent cellular cytotoxicity (ADCC).

The current invention alleviates the problems of the prior art by providing a combination of agents for use in the treatment of MM in a subject.

### SUMMARY OF THE INVENTION

The present invention is based on the unexpected discovery that a desialylation agent and an anti-CD38 antibody, in combination, significantly increases NK cell mediated ADCC against multiple myeloma cells. The resulting NK cell mediated ADCC is unmatched by either of these treatments alone.

An aspect of the invention provides an effective amount of a desialylation agent and an effective amount of a CD38 targeting agent for use in the treatment of a CD38 positive disease.

In an embodiment, the disease is selected from the group comprising a haematological malignancy such as large B cell lymphoma, T cell acute lymphoblastic leukaemia (T-ALL), acute myeloid leukaemia (AML), chronic lymphocytic leukaemia (CLL) and multiple myeloma.

Preferably, the disease is multiple myeloma. In one embodiment, the desialylation agent and the CD38 targeting agent, e.g. the anti-CD38 antibody, are in the form of a conjugate.

In one embodiment, said agents for use in treatment further comprises an effective amount of a modified NK cell.

In one embodiment, said agents for use in treatment, further comprises an effective amount of an agent that increase the expression of CD38 on the cell surface of the multiple myeloma cell.

An aspect of the invention provides an effective amount of a desialylation agent, an effective amount of an agent that increases the expression of CD38 on the cell surface of a cell and an effective amount of a CD38 targeting agent, for use in the treatment of a CD38 positive disease, e.g. multiple myeloma.

An aspect of the invention provides an effective amount of a desialylation agent, an effective amount of a modified NK cell and an effective amount of a CD38 targeting agent, for use in the treatment of a CD38 positive disease, e.g. multiple myeloma.

An aspect of the invention provides an effective amount of a desialylation agent, an effective amount of an agent that increases the expression of CD38 on the cell surface of a cell, an effective amount of a modified NK cell and an effective amount of a CD38 targeting agent, for use in the treatment of a CD38 positive disease, e.g. multiple myeloma.

The CD38 targeting agent in any aspect of the invention may be an anti-CD38 antibody.

The CD38 targeting agent may be an immune cell that expresses an antigen receptor targeting CD38. The immune cell may be an NK cell. The immune cell may be a T cell.

Preferably, the desialylation agent in any aspect of the invention may be selected from the group comprising a sialidase enzyme and sialyltransferase inhibitor (SIA).

In an embodiment, the modified NK cell in any aspect of the invention may be an NK cell that does not express CD38 or has reduced expression of CD38 compared to an unmodified NK cell.

An aspect of the invention provides a method of treating a CD38 positive disease, said method comprising the steps of:

administering an effective amount of a desialylation agent and an effective amount of a CD38 targeting agent, to a subject in need thereof.

The disease may be multiple myeloma.

The CD38 targeting agent may be an anti-CD38 antibody.

The method may further comprise administering an effective amount of a modified NK cell to said subject.

The method may further comprise administering an effective amount of an agent that increases the expression of CD38 on the surface of the multiple myeloma cell.

The desialylation agent and the anti-CD38 antibody may be in the form of a conjugate.

A conjugate comprising a desialylation agent and a CD38 targeting agent is provided.

A conjugate comprising a desialylation agent and an anti-CD38 antibody is provided.

A composition comprising the conjugate is provided.

A composition comprising a desialylation agent, a CD38 targeting agent and a modified NK cell is provided.

A composition comprising a desialylation agent, a CD38 targeting agent and an agent that increases the expression of CD38 is provided.

A composition comprising a desialylation agent, a CD38 targeting agent, a modified NK cell and an agent that increases the expression of CD38 is provided.

Definitions and General Preferences

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

When used herein the term “antibody-dependent cellular cytotoxicity (ADCC)” is a mechanism of cell mediated immune defence whereby an effector cell actively lyses a target cell, whose membrane surface antigens have been bound by specific antibodies, in an Fc receptor dependent manner. The effector cell may be a natural killer cell.

When used herein the term “natural killer (NK) cell” is a type of cytotoxic lymphocyte involved in the innate immune system of a subject.

When used herein the term “desialylation agent”, refers to any agent that can remove or reduce the amount of sialic acid on a cell surface, preferably a multiple myeloma cell surface. Methods of determining desialylation or expression or presence of sialic acid on the cell surface are known to the person skilled in the art. For example, sialic acid expression may be measured on the cell surface by using specific sialic acid-binding lectins in a flow cytometry screening assay. For example, MAL-II can bind to α2,3-linked sialic acids while the lectin SNA can bind to α2,6-linked sialic acids. The specificity of these lectins is based on the type of linkage of the sialic acid to the underlying glycan. These lectins can be used to measure the expression, or lack of, sialic acids, before and/or after desialylation using sialidases or sialyltransferase inhibitors. Baum et al, Journal of Biological Chemistry, 271, 10793-10799, May 3, 1996, the content of which is incorporated herein, provides an example of a method using SNA to measure α2,6-linked sialic acids.

When used herein the term “CD38 targeting agent” refers to an agent that is targeted or recognises CD38 on the surface of a cell, preferably a multiple myeloma cell. Typically, it is a CD38 binding agent, i.e. one that binds to CD38. It may be direct or indirect binding. It may be specific for CD38. The agent may be selected from, but not limited to, a peptide, a small molecule, peptide mimic, antagonist, an antibody and a cell such as an immune cell. Typically, the agent has a CD38 targeting or binding domain. One example is a chimeric antigen receptor for CD38. One example is a CD38 CAR-NK cell.

When used herein the term “an agent that increases the expression of CD38” is any agent that upregulates or increases the expression of CD38 on the surface of a cell relative to the wildtype or unmodified cell. Such agents are known in the field. Methods to determine CD38 expression are known to the person skilled in the art and any such method may be used. One method is using flow cytometry to determine CD38 expression on MM cells, such as the method described by Inger S. Nijhof et al., Blood (2016) 128 (7) 959-970.

When used herein the term “CD38 antibody” or “anti-CD38 antibody”, is an antibody, preferably a monoclonal antibody, that targets CD38 which is a cell surface glycoprotein. CD38 is expressed on myeloma cells but is also expressed at low levels on normal white blood cells. Anti-CD38 antibodies are described, for example, in Int'l Pat. Pub. No. WO2008/037257, Int'l Pat. Pub. No. WO2008/047242 and Int'l Pat. Pub. No. WO2007/042309.

The term “antibody” refers to an antibody in any form, including but not limited to monoclonal, polyclonal, humanized or human form, or antibody fragments, preferably that bind to CD38. A humanized antibody may comprise portions of immunoglobulins of different origin. For example, at least one portion can be of human origin. Methods of preparing immunoglobulins, immunizing with antigens, and polyclonal and monoclonal antibody production can be performed using known suitable techniques.

As used herein, the term “treatment” or “treating” refers to an intervention (e.g. the administration of an agent to a subject) which cures, ameliorates or lessens the symptoms of a condition or disease or removes (or lessens the impact of) its cause(s). In this case, the term is used synonymously with the term “therapy”. Additionally, the terms “treatment” or “treating” refers to an intervention (e.g. the administration of an agent to a subject) which prevents or delays the onset or progression of a disease or reduces (or eradicates) its incidence within a treated population. The term treatment is used synonymously with the term “prophylaxis”.

As used herein, “an effective amount” or “a therapeutically effective amount” of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. In preferred embodiments, the subject is a human.

When used herein, the term “composition” should be understood to mean something made by the hand of man, and not including naturally occurring compositions. Compositions may be formulated in unit dosage form, i.e., in the form of discrete portions containing a unit dose, or a multiple or sub-unit of a unit dose.

When used herein, the term “modified NK cell”, means an NK cell that has been genetically changed or modified compared to an unmodified NK cell. In this instance, the NK cell may be genetically modified such that the cell does not express or has reduced expression of CD38.

As used herein, the term “genetically modified” means genetically engineered using recombinant DNA technology.

As used herein the term “a CD38 positive disease” is a condition or disease which is characterised by an expression of CD38 on the surface of cells, preferably cancer or tumour cells, or increased or overexpression of CD38 on the surface of cells, preferably cancer or tumour cells, compared with normal healthy cells, in a subject. Such diseases are known to the person in the art and are selected from, but not limited to, the group comprising a haematological malignancy, including large B cell lymphoma, T cell acute lymphoblastic leukaemia (T-ALL), acute myeloid leukaemia (AML), chronic lymphocytic leukaemia (CLL) and multiple myeloma, Burkitt's lymphoma, follicular lymphoma, mantle-cell lymphoma, or combinations thereof. This can be used interchangeably with the term “CD38 positive malignancy”.

“CD38” refers to the human CD38 protein (UniProtKB accession no. P28907) (synonyms: ADP-ribosyl cyclase 1, cADPr hydrolase 1, cyclic ADP-ribose hydrolase 1).

### BRIEF DESCRIPTION OF THE INVENTION

The invention will now be described with reference to the accompanying Figures in which;

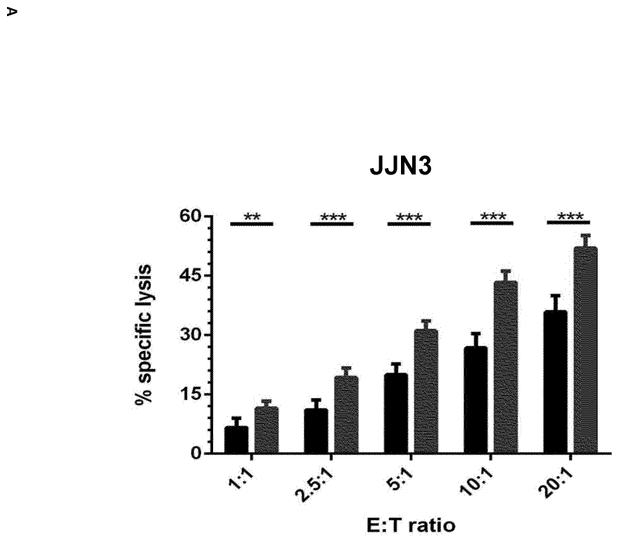

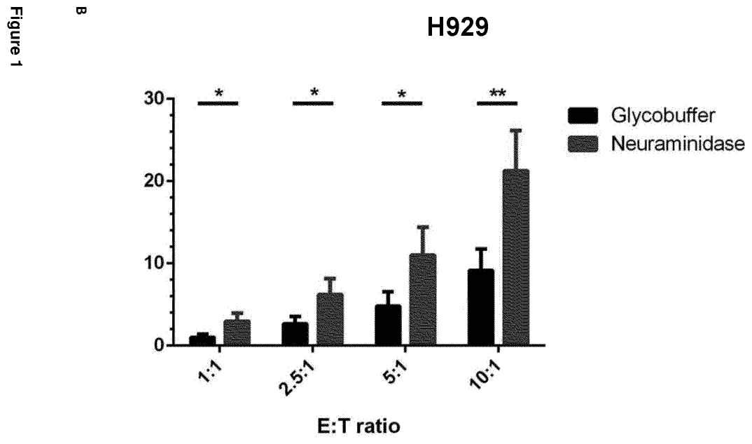

FIG. 1: Desialylation of MM cells lines JJN3 (A) and H929 (B) with neuraminidase results in enhanced IL-2 activated primary NK cell mediated cytotoxicity. N=6 for JJN3, N=7 for H929, graph represents mean+SEM, co-cultures carried out for 4 hrs, data analysed using student's paired t-test, *−p<0.05, **−p<0.01, ***−p<0.001.

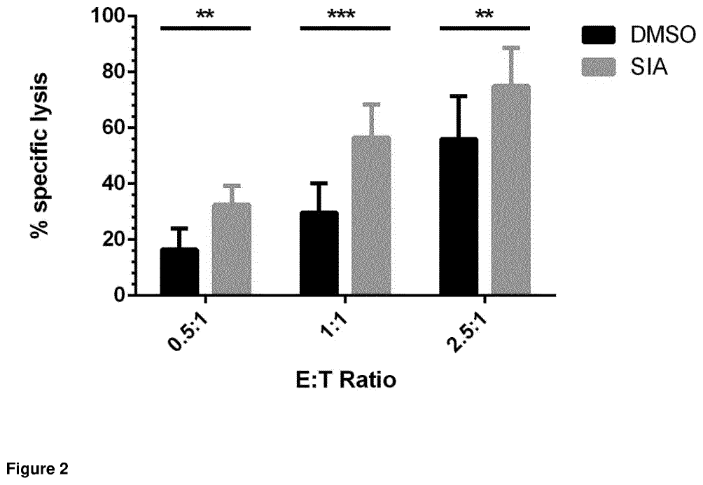

FIG. 2: Desialylation of MM cell line H929 with the sialyltransferase inhibitor 3Fax-Peracetyl Neu5Ac (SIA) results in enhanced expanded primary NK cell-mediated cytotoxicity. N=5, graph represents mean+SEM, co-cultures carried out for 4 hrs, data analysed using student's paired t-test, **−p<0.01, ***−p<0.001.

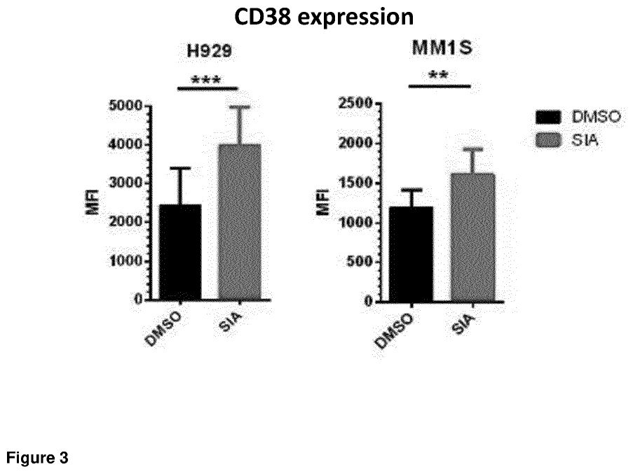

FIG. 3: Desialylation of MM cell lines MM1S and H929 using SIA unmasks CD38. N=5 for each, MFI—mean fluorescence intensity, graph represents mean+SD, **−p<0.01, ***−p<0.001.

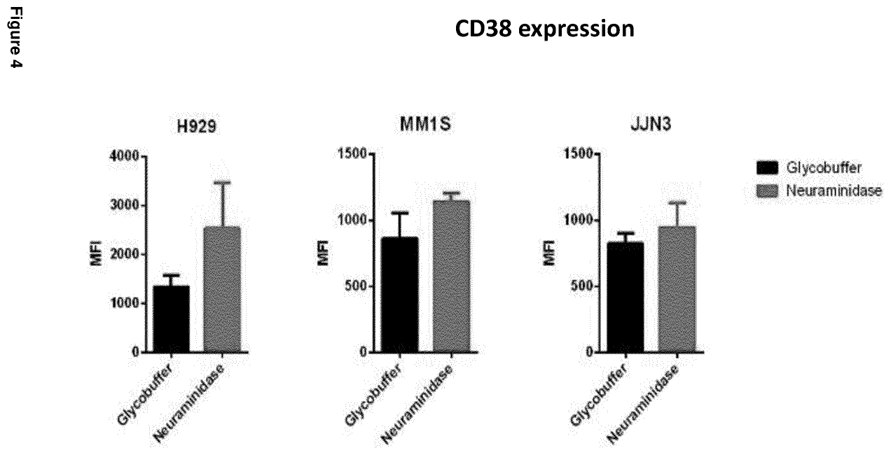

FIG. 4: Desialylation of MM cell lines using the sialidase neuraminidase unmasks CD38. N=3 for each, MFI—mean fluorescence intensity, graph represents mean+SD.

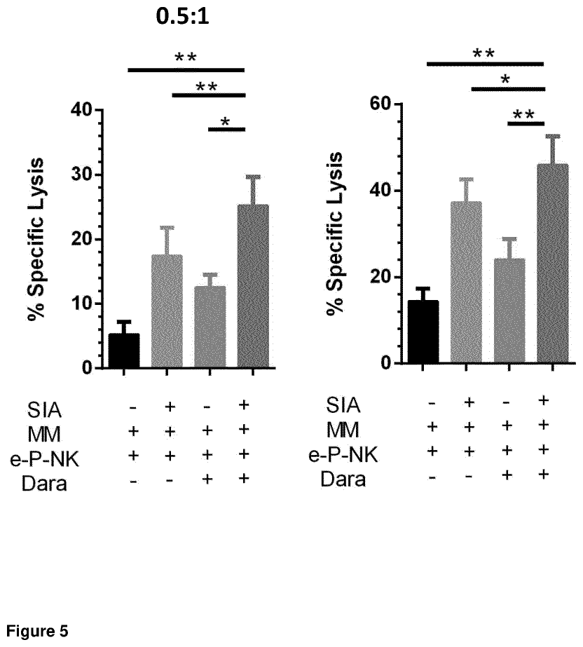

FIG. 5: Treatment of MM cell line H929 with SIA+Daratumumab results in enhanced expanded primary NK cell-mediated cytotoxicity then either SIA or Dara alone. N=5, graph represents mean+SEM, co-cultures carried out for 4 hrs, data analysed using one-way ANOVA, *−p<0.05, **−p<0.01.

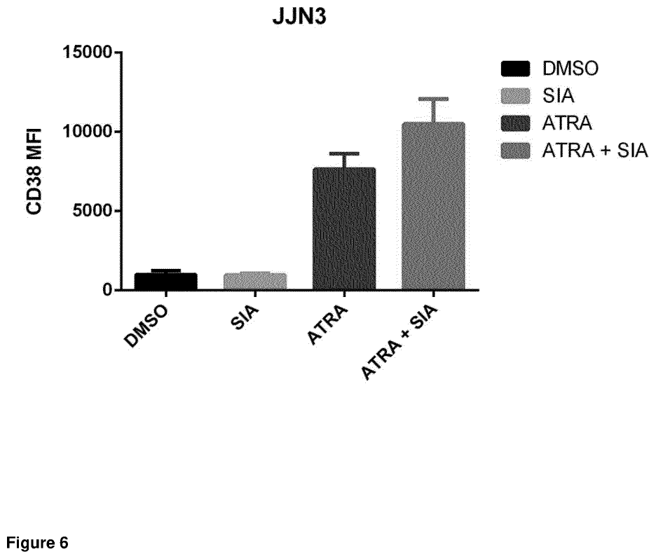

FIG. 6: ATRA+SIA treatment of JJN3 upregulates CD38 expression to a level higher than ATRA or SIA treatment alone. N=3, graph represents mean+SD, JJN3 treated with SIA, ATRA or SIA+ATRA for 72 hours prior to expression measurement.

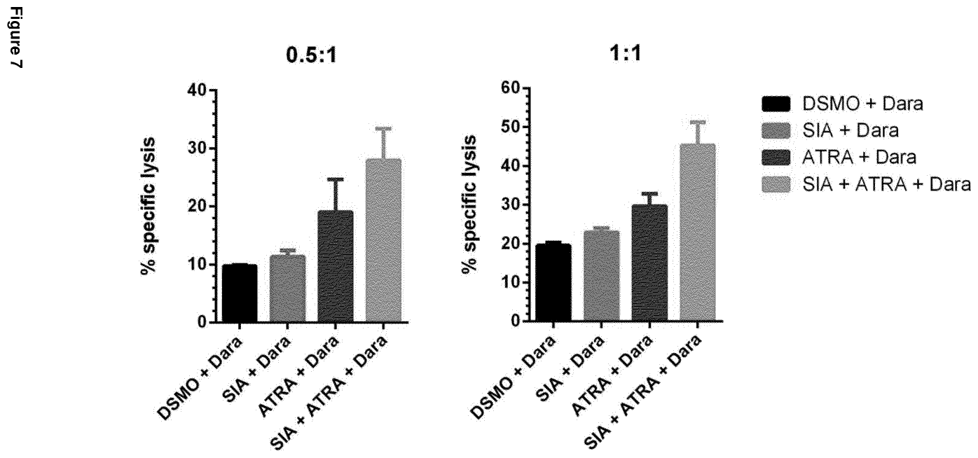

FIG. 7: JJN3 cells treated with ATRA, SIA and Dara are more sensitive to expanded NK cell-mediated killing than ATRA+Dara or SIA+Dara alone. N=2, graph represents mean+SD, JJN3 treated with SIA, ATRA or SIA+ATRA for 72 hours prior to cytotoxicity assay.

### DETAILED DESCRIPTION OF THE INVENTION

The details of one or more embodiments of the invention are set forth in the description below. Other features, objects and advantages of the invention will be apparent form the description and the claims.

The current inventors have surprisingly shown that targeted removal of the sialic acid layer on the surface of multiple myeloma cells serves to unmask an amount of the glycoprotein CD38 and surprisingly, results in potentiated primary NK cell activity against the cell. The NK cell may be chimeric antigen receptor (CAR)-NK. The removal may be complete removal or partial removal of sialic acid layer. This results in the increased expression or availability of CD38 on the surface of the multiple myeloma cell.

Not to be bound by theory, the current inventors consider that the increase of NK cell cytotoxicity against the tumour cells that have been desialylated may be due to the removal of sialic acid derived ligands for inhibitory sialic acid-binding immunoglobulin-like lectins (Siglecs, Siglec-7 and Siglec-9 in the case of NK cell; these are inhibitory receptors expressed by NK cells).

The current inventors further surprisingly discovered that targeted removal of the sialic acid layer on the surface of multiple myeloma cells in combination with administration of an anti-CD38 antibody, resulted in a level of NK cell mediated ADCC is unmatched by either of these treatments alone.

In a notable embodiment the current invention provides the use of the desialylation agent or method of the invention to increase CD38 expression to enhance cytotoxicity of CAR-NK cell targeting CD38 on a tumour cell.

The current invention provides an effective amount of a desialylation agent for use in the treatment of a CD38 positive disease in a subject. The disease may be multiple myeloma in an embodiment. The desialylation agent is for use in combination with an effective amount of a CD38 targeting agent. The desialylation agent may be referred to as a first composition and the CD38 targeting agent may be referred to as a second composition in aspects of the current invention

It has been surprisingly discovered that this specific combination achieves superior results than would be expected with either of these treatments alone. In other words, there is a synergistic effect. The level of NK cell mediated ADCC is unmatched by either of these treatments alone.

Again, not to be bound by theory, increased NK cell activity against multiple myeloma cells with this specific combination is due to the unmasking of CD38 antigen (i.e. increasing CD38 availability or expression) and subsequent binding of the antibody to CD38, leading to activation of the NK cell through the Fc chain-binding receptor CD16 in a process known as antibody-dependent cell-mediated cytotoxicity (ADCC). It is considered that the enhanced ADCC is due to a combination of enhanced availability of CD38 and reduced NK cell inhibition.

The CD38 targeting agent may be a CD38 binding agent. The CD38 binding agent may be an anti-CD38 antibody. Typically, the anti-CD38 antibody in any aspect of the invention is a monoclonal antibody. The antibody may be daratumumab. The antibody may be a bispecific antibody, a polyclonal antibody, a chimeric antibody, a human antibody, a humanized antibody, a hybrid antibody, an antigen binding fragment thereof. The antibody may be a conjugated antibody, e.g. conjugated with a toxin or sialidase. The toxin may be one that targets tumour cells which express an antigen not expressed by normal healthy cells.

The desialylation agent and the anti-CD38 antibody may be in the form of an agent-antibody conjugate.

The CD38 targeting agent may be a cell that expresses an antigen receptor targeting CD38. The cell may be an immune cell, such as a NK cell or a T cell. The cell may be genetically engineered to express the antigen receptor. The cell may be linked to one or more co-stimulatory domains and/or stimulatory domains. The antigen receptor may be a CD38 antigen receptor or a chimeric antigen receptor. Examples include a chimeric antigen receptor (CAR)-NK or CAR-T cells, which are cells that contain a chimeric antigen receptor. The CAR-cell contains a receptor typically made up of an antigen, e.g. CD38, binding scFv (derived from the variable regions of the heavy and light chains of a monoclonal antibody or only the heavy chains of a monoclonal antibody), linked to one or more co-stimulatory domains (e.g. CD28, 4-1 BB, DAP10, DAP12 etc) and a stimulatory domain (e.g. CD3 zeta). In use, on recognition and binding to a target cell (e.g. CD38 positive MM cell in the case of a CD38 CAR-T or CD38 CAR-NK), this leads to activation of the CAR-T or CAR-NK, followed by degranulation and lysis of the target cell. The CD38NK cell is capable of expressing more CD38 than T cells. The antigen receptor may be such that it will only recognise MM cells with very high expression of CD38 compared to normal healthy cells. In this way it does not recognise cells with moderate expression of CD38 such as NK cells. This type of cell has been reported by Drent E, et al. Mol Ther. 2017 Aug. 2; 25(8)1946-1958, the content of which is incorporated herein by reference. CAR-T cells are disclosed in Kloess et al., Transfus Med Hemother. 2019 February; 46(1): 4-13, the content of which is incorporated herein by reference.

Any suitable schedule of administration may be used. The desialylation agent may be administered to the subject before the CD38 targeting agent. The desialylation agent may be administered after the CD38 targeting agent to the subject. The desialylation agent may be administered simultaneously with the CD38 targeting agent to the subject. In other words, the agent and targeting agent may be co-administered or administered at separate times. For instance, they may be administered on alternate days, on the same day at different times, or on different days. The agents or drugs may be administered together. One suitable way of achieving this is the provision of both agents in unit dose form, for example a formulation comprising the two agents. For example, a conjugate. One example is a desialylation agent-antibody conjugate, in the form of a CD38 antibody conjugated with the desialylation agent. An example of a sialidase-antibody conjugate is described in Xiao Han, et al, PNAS Sep. 13, 2016, 113 (37) 10304-10309, the content of which is incorporated herein by reference. They may be admixed or kept in separate parts of the unit dose. The time between administration of the agent and the CD38 targeting agent, i.e. the first composition and the second composition, may be one suitable for the first composition to have the desired or suitable effect, i.e. removal or reduction of the sialic acid layer of the MM cell. Administration or treatment may be repeated. The time of treatment is that sufficient to treat the disease.

It will be appreciated that the desialylation agent may be any known suitable agent in the art. The desialylation agent may be selected from the group comprising, but not limited to, a sialidase enzyme, for example neuraminidase, or a sialytransferase inhibitor (SIA). Neuraminidases (also referred to as “neura”) functions by removing sialic acids. Neuraminidases are known in the art and any suitable neuraminidase may be used. For example, the neuraminidase may be one isolated from bacteria, for instance, Vibrio cholerae. The sialyltransferase inhibitor may be 3Fax-Peracetyl Neu5Ac.

The subject is one with multiple myeloma disease. Multiple myeloma may be at stage or category. The subject may be one with MM at initial presentation. The subject may have relapsed and/or refractory MM. The MM may be at an advanced stage. The subject may be any age and/or fragility. The MM may be one associated with or having hypersialylated multiple myeloma cell surface. Increasing sialylation is a general feature of MM disease progression. This can be detected by the use of specific lectins that recognise α 2,3 and α 2,6 linked (MAL, SNA) sialic acids, for example. Currently, there is no therapeutic approach specifically targets the hypersialylated MM cell surface available to patients. The subject may be one that has already received treatment, e.g. treatment with an anti-CD38 antibody.

The subject may be one with a low level of CD38 expression on the cell surface compared to a healthy subject, i.e. one without multiple myeloma. The low level may be no expression or substantially no expression.

As previously discussed, there can be a significant reduction in CD38 expression on myeloma cells in multiple myeloma patients following treatment with a CD38 antibody, e.g. Daratumumab. Targeted desialylation could lead to increased CD38 availability/expression in these subjects.

The NK cells may be the subjects host NK cells within their immune system.

Typically, the agent(s)for use of the invention may be formulated for oral delivery or injection. The agent(s) may be a composition comprising the agent. The composition may comprise one or more carriers. The carrier in the pharmaceutical composition must be “acceptable” in the sense of being compatible with the active ingredient of the formulation (and preferable, capable of stabilising it) and not deleterious to the subject to be treated. The method of introduction may be the same for teach agent or it may be different. Methods of introduction or administration include but are not limited to intradermal, intramuscular, intraperitoneal, intravenous, subcutaneous, intranasal, epidural, intranasal, intracerebral, and oral routes. Administration may be by any convenient route, for example by infusion or bolus injection, by absorption through epithelial or mucocutaneous linings (e.g., oral mucosa, rectal and intestinal mucosa, etc). Administration can be systemic or local. The agent(s) may be formulated for slow release or sustained release. Preferably, the agent(s) is formulated for intravenous delivery, optionally as a free drug. Preferably, the CD38 binding agent is formulated for intravenous or subcutaneous administration. In an embodiment, the agent may be conjugated to the CD38 binding agent. The agent(s) may be administered in a targeted fashion, for example by a nanoparticle or a liposome. A composition for oral administration can be any orally acceptable dosage form including, but not limited to, capsules, tablets, emulsions and aqueous suspensions, dispersions and solutions.

The medical use of the invention may further comprise an effective amount of an agent that upregulates CD38 expression. In this aspect, the invention provides an effective amount of a desialylation agent, an effective amount of an agent that upregulates CD38 expression, and an effective amount of a CD38 targeting agent, for use in the treatment of multiple myeloma in a subject.

The agent that upregulates CD38 expression may be any suitable agent and such agents are known in the art. It may be a composition as described herein. An example is all-trans retinoic acid (ATRA), also known as tretinoin. Tretinoin, is currently used to treat acute promyelocytic leukaemia (APL) in a capsule form, administered orally. The agent may be a pan-histone deacetylase inhibitor, e.g. Panobinostat. Garcia-Guerrero Estefania et al., Blood 2017, 129, (25) 3386-3388 discloses panobinostat, the content of which is incorporated herein by reference.

The formulation and route of administration may be as disclosed herein in relation to the agent and/or antibody. The agent may be a composition comprising the agent. The agent may be formulated for oral administration. The agent may be administered in a liposome or nanoparticle. Liposomes encapsulating such agents are disclosed for example in Estey E, Blood (1996) 87 (9) 3650-3654, the content of which is incorporated herein by reference.

The schedule of administration may be any suitable schedule as disclosed herein. The agent may be administered to the subject before the CD38 targeting agent. The agent may be administered to the subject before the desialylation agent and the CD38 binding agent. This will provide sufficient upregulation of CD38 expression on MM cells of the subject.

The medical use of the invention may further comprise an effective amount of a modified NK cell. In this aspect, the invention provides an effective amount of a desialylation agent, an effective amount of a modified NK cell, and an effective amount of a CD38 targeting agent, for use in the treatment of multiple myeloma in a subject.

The modified NK cell may be a NK cell that has no or reduced expression of CD38 compared to an unmodified cell. These cells may be referred to as CD38 deficient NK cells. Modified NK cells and method to prepare same are known in the art. An example is disclosed in Woan et al., Blood, volume 132, issue supplement 1, Nov. 29, 2018, the content of which is disclosed herein by reference.

The NK cells may be primary NK cells (of donor or autologous origin), NK cells derived from a pluripotent stem cell or an NK cell line. Ideally, these NK cells should have been genetically modified to reduce the expression of CD38. This could be achieved using gene editing technologies, such as, but not limited to CRIPSR/Cas9, TALENS, shRNA or siRNA.

The formulation and route of administration may be as disclosed herein in relation to the agent and/or antibody. The modified NK cell may be a composition comprising the modified NK cell. The modified NK cell may be formulation for intravenous injection or infusion to a subject.

The schedule of administration may be any suitable schedule. In an embodiment, the modified NK cell is administered to the subject concurrently or subsequent to treatment with the desialylation agent. In an embodiment, the modified NK cell is administered to the subject concurrently or subsequent to the CD38 targeting agent. The NK cells may be administered as a single or repeated dose or infusion. The NK cells may be administered in conjunction with the desialylation agent and CD38 targeting agent.

The invention also provides an effective amount of a desialylation agent, an effective amount of an agent that upregulates CD38 expression, an effective amount of a modified NK cell and an effective amount of a CD38 binding agent, for use in the treatment of a CD38 positive disease, such as multiple myeloma, in a subject.

Also provided by the current invention is a method of treating a CD38 positive disease. Also provided by the current invention is a method of treating multiple myeloma in a subject. All features and embodiments discussed herein in relation to the use of the invention also apply to the disclosed method of treatment. The subject is one in need thereof.

The methods of the invention comprise administering an effective amount of a desialylation agent and an effective amount of a CD38 targeting agent, to the subject.

The method may further comprise administering an effective amount of a modified NK cell to said subject. The NK cell may be administered before, concurrently or after the desialylation agent.

The method may further comprise administering an effective amount of an agent that increases the expression of CD38 on the surface of the tumour cell, e.g. a multiple myeloma cell. The agent may be administered before or concurrently with the CD38 targeting agent. The desialylation agent and the CD38 targeting agent may be in the form of a conjugate. The desialylation agent, the CD38 targeting agent, the modified NK cell and/or the agent that increases the expression of CD38 on the surface of the multiple myeloma cell, may be administered simultaneously or at separate times. Any suitable schedule for administration is considered herein.

A method of enhancing CD38 expression is also provided, said method comprising administration of a desialylation agent to a subject in need thereof.

The accompanying Examples, show that specific targeted removal of the dense sialic acid layer on the MM cell surface results not only in potentiated primary NK cell activity against a myriad of MM cell lines (FIGS. 1 and 2), but also uncovers previously masked epitopes, in this case the glycoprotein CD38, a well validated target in the treatment of MM (FIGS. 3 and 4).

The inventors have seen that the targeted desialylation of the MM cell surface results in strongly increased primary NK cell-mediated killing of the MM cell line H929. Furthermore, the inventors have shown that H929 MM cells treated with both Daratumumab (Dara, anti-CD38) and 3Fax-Peracetyl Neu5Ac (SIA, sialyltransferase inhibitor) were more sensitive to expanded primary NK cell-mediated immunosurveillance than either Dara or SIA alone (FIG. 5).

Finally, JJN3 MM cells treated with ATRA (an agent known to upregulate CD38 via a transcriptional mechanism) and SIA were more readily targeted by expanded primary NK cells than JJN3 cells treated with ATRA or SIA individually (FIG. 7). This increase was due to enhanced ADCC of the JJN3 cells by NK cells because of the higher levels of CD38 expression of JJN3 treated with the combination of ATRA and SIA.

The masking of the CD38 antigen by the hypersialylated MM cell surface limits ADCC-mediated by NK cells, the full therapeutic value of an anti-CD38 monoclonal antibody appears to be when in combination with desialylation of the MM cell surface, revealing any previously masked CD38 antigen. Moreover, the inventors have shown that the combination of an agent that upregulates CD38 expression and desialylation has an additive effect. This suggest a role for this combination in patients with low levels of CD38 expression.

Targeted desialylation may also enhance complement dependent cytotoxicity (CDC), which is an important mechanism of action of some anti-CD38 monoclonal antibodies, such as Daratumumab.

### EXAMPLES

### Example 1

Analysis of NK Cell Mediated Cytotoxicity and CD38 After Treatment with Sialyltransferase Inhibitor or Neuraminidase

Methodology

Desialylation: Target cells were cultured with 300 μM 3Fax-Peracetyl Neu5Ac (SIA, sialyltransferase inhibitor) for 72 hrs prior to use or treated with Neuraminidase (Neura, sialidase) for 45 mins@37° C. prior to use.

Co-cultures were carried out with either expanded (Miltenyi method) primary NK cells or IL-2 activated primary NK cells, both originally isolated from peripheral blood supplied by healthy donors and H929 and JJN3 target myeloma cell lines. The Miltenyi method is known in the art and described at the following link:https://www.miltenvibiotec.com/IE-en/products/macs-cell-culture-and-stimulation/media/other-media/nk-macs-medium.html and https://www.miltenyibiotec.com/upload/assets/IM0020747.PDF.

Expanded NK cells are reported to have altered receptor profiles, which is reported to happen throughout the course of expansions. The current inventors have observed that expanded NK cells are more potent against target cells than IL-2 activated NK cells, which are isolated form the blood sample and simply incubated overnight in standard RPMI-1640 media+IL-2.

Both types of primary NK cell were cultured with the same amount of IL-2 (500 U/trip as is standard for primary NK cell work. MM1S, H929 and JJN3 were treated with either SIA or Neura as described above and stained with an anti-CD38 antibody (BD Pharmingen-564498) for 30 mins on ice prior to being screened for expression using flow cytometry.

Propidium Iodide was used in flow cytometry to identify dead cells. MM cells were separated from NK cells by staining NK cells with CFSE cell tracking dye prior to initiating the co-culture.

% specific lysis (reported in all our figures) was calculated using the following formula:

Total cell death−basal cell death/100−basal cell death×100=%

Cell death here applies to target cells (e.g. JJN3 and H929)

Results

Desialylation, using either a sialidase or a sialyltransferase inhibitor, strongly enhances NK cell activity against MM cell lines (FIGS. 1 and FIG. 2).

H929 and MM1S MM cells treated with a sialyltransferase inhibitor and sialidase show increased expression of the CD38 compared to non-treated controls (FIGS. 3 and 4, respectively).

### Example 2

Analysis of NK cell mediated cytotoxicity and CD38 after treatment with sialyltransferase inhibitor and Daratumumab or sialyltransferase inhibitor and ATRA.

Methodology

JJN3 MM cells were treated with 300 μM SIA and 10 nM ATRA (all-trans retinoic acid), or 300 μM SIA, 10 nM ATRA and DMSO individually for 72 hrs@37° C. After 72 hours, the cells were collected, washed with normal culture media once and then treated with Daratumumab at a concentration of 10 μg/ml for 30 mins at room temperature.

Some cells from all treatment conditions were not treated with Daratumumab and were instead screened for CD38 expression using an anti-CD38 antibody.

After 30 mins treatment with Daratumumab, cells were co-cultured with primary expanded NK cells (Miltenyi method) for 4 hours at either 0.5:1 or 1:1 E:T (Effector:Target) ratios.

Results

ATRA strongly up-regulates CD38 expression on MM cells. The current inventors have combined ATRA treatment with desialylation, using the sialyltransferase inhibitor, 3Fax-Peracetyl Neu5Ac, to show that even when significantly up-regulated, CD38 is still masked considerably by sialic acids.

H929 MM cells treated with both Daratumumab (Dara, anti-CD38) and 3Fax-Peracetyl Neu5Ac (SIA, sialyltransferase inhibitor) were more sensitive to expanded primary NK cell-mediated immunosurveillance than either Dara or SIA alone (FIG. 5).

Furthermore, when treated with ATRA and SIA, expression of CD38 on JJN3 treated with both ATRA and SIA was higher than on JJN3 treated with either ATRA or SIA alone (FIG. 6).

This represents a further means to enhance ADCC mediated by NK cells (FIG. 5) as more CD38 antigen is exposed when cells are treated with both ATRA and SIA together than either of the two alone (see FIG. 6).

Finally, JJN3 MM cells treated with ATRA and SIA were more readily targeted by expanded primary NK cells than JJN3 cells treated with ATRA or SIA individually (FIG. 7). This increase was due to enhanced ADCC of the JJN3 cells by NK cells because of the higher levels of CD38 expression of JJN3 treated with the combination of ATRA and SIA.

1. An effective amount of a desialylation agent and an effective amount of a CD38 targeting agent, for use in the treatment of a CD38 positive disease.

2. The desialylation agent and CD38 targeting agent for use of claim 1, in which the disease is selected from the group comprising a haematological malignancy, including large B cell lymphoma, T cell acute lymphoblastic leukaemia (T-ALL), acute myeloid leukaemia (AML), chronic lymphocytic leukaemia (CLL) and multiple myeloma.

3. The desialylation agent and CD38 targeting agent for use of claim 2, in which the disease is multiple myeloma.

4. The desialylation agent and CD38 targeting agent for use any one of the preceding claims, further comprising a modified NK cell.

5. The desialylation agent and CD38 targeting agent for use of any one of the preceding claims, further comprising an effective amount of an agent that increases the expression of CD38 on the surface of a cell.

6. The desialylation agent and CD38 targeting agent for use of any one of the preceding claims, wherein the desialylation agent is selected from the group comprising a sialidase enzyme and sialytransferase inhibitor (SIA).

7. The desialylation agent and CD38 targeting agent for use of any one of the preceding claims, in which the CD38 targeting agent is selected from the group comprising a peptide, a small molecule, a peptide mimic, an antagonist, an antibody and an immune cell.

8. The desialylation agent and CD targeting agent for use of claim 7, wherein the agent is a chimeric antigen receptor for CD38.

9. The desialylation agent and CD38 targeting agent for use of claim 7, wherein the antibody is a monoclonal antibody.

10. The desialylation agent and CD38 targeting agent for use of claim 7, wherein the CD38 targeting agent is an immune cell that expresses an antigen receptor targeting CD38.

11. The desialylation agent and CD38 targeting agent for use of claim 10, in which the immune cell is an NK cell or a T cell.

12. The desialylation agent and CD38 targeting agent for use of any one of the preceding claims, wherein the desialylation agent and the CD38 binding agent are in the form of a conjugate.

13. The desialylation agent and CD38 targeting agent for use of any one of claims 4 to 12, wherein the modified NK cell is an NK cell that does not express CD38 or has reduced expression of CD38 compared to an unmodified cell.

14. An effective amount of a desialylation agent, an effective amount of an agent that increases the expression of CD38 on the surface of a cell, and an effective amount of a CD38 targeting agent, for use in the treatment of a CD38 positive disease.

15. An effective amount of a desialylation agent, an effective amount of a modified NK cell and an effective amount of a CD38 targeting agent for use in the treatment of a CD38 positive disease.

16. An effective amount of a desialylation agent, an effective amount of a modified NK cell, an effective amount of an agent that increases the expression of CD38 on the surface of a multiple myeloma cell and an effective amount of a CD38 binding agent, for use in the treatment of a CD38 positive disease.

17. A conjugate comprising a desialylation agent and a CD38 targeting agent.

18. The conjugate of claim 17, in which the CD38 targeting agent is an anti CD38 antibody.

19. A composition comprising the conjugate of claim 17 or 18.

20. The composition of claim 19, further comprising an effective amount of an agent that increases the expression of CD38 on the surface of a cell.

21. The composition of claim 19 or 20, comprising an effective amount of a modified NK cell that does not express CD38 or has reduced expression of CD38 compared to an unmodified cell.

22. A method for the treatment of a CD38 positive disease in a subject, said method comprising administration of a desialylation agent and a CD38 targeting agent, to said subject.

23. The method of claim 22, in which the disease is multiple myeloma.

24. The method of claim 22 or 23, comprising administration of a modified NK cell.

25. The method of any one of claims 22 to 24, comprising administration of an agent that increases the expression of CD38 on the surface of a cell.

26. The method of any one of claims 22 to 25, wherein the desialylation agent is selected from the group comprising a sialidase enzyme and sialytransferase inhibitor (SIA).

27. The method of any one of claims 22 to 26, wherein the CD38 targeting agent is selected from the group comprising a peptide, a small molecule, a peptide mimic, an antagonist, an antibody and an immune cell.
